(19) United States
(12) Patent Application Publication
(10) Pub. No.: US 2019/0090768 A1
(43) Pub. Date: Mar. 28, 2019

(54) VITAL SIGN WAVEFORM DISPLAY APPARATUS, VITAL SIGN WAVEFORM DISPLAY METHOD, PROGRAM, AND STORAGE MEDIUM

(71) Applicant: NIHON KOHDEN CORPORATION, Shinjuku-ku, Tokyo (JP)

(72) Inventor: Yasushi OIKAWA, Shinjuku-ku, Tokyo (JP)

(21) Appl. No.: 15/768,871

(22) PCT Filed: Oct. 17, 2016
(86) PCT No.: PCT/JP2016/004607

§ 371 Date: Apr. 17, 2018

(30) Foreign Application Priority Data
Oct. 19, 2015 (JP) 2015-205383

(51) Int. Cl. A61B 5/044; A61B 5/00

(52) U.S. Cl. CPC A61B 5/044; A61B 5/7225; A61B 5/742

(57) ABSTRACT

A vital sign waveform display apparatus includes: an acquiring section configured to acquire vital sign waveform data representing a plurality of vital sign waveforms; a generating section configured to generate the plurality of vital sign waveforms, based on the acquired vital sign waveform data; and a display controller configured to display the generated vital sign waveforms side by side on a display screen. The display controller highlights a first vital sign waveform when the first vital sign waveform is selected from the vital sign waveforms displayed on the display screen, and then the display controller highlights the first vital sign waveform and a second vital sign waveform when the second vital sign waveform is selected from the vital sign waveforms displayed on the display screen.

### TECHNICAL FIELD

The present disclosure relates to a vital sign waveform display apparatus, and further relates to a vital sign waveform display method, a program for executing the method, and a storage medium storing the program.

### BACKGROUND

JP-A-4-150832 discloses that a plurality of electrocardiogram (ECG) waveforms that are examples of a vital sign waveform are analyzed while being displayed side by side on one display screen.

In the analytical technique, as the number of ECG waveforms that are displayed side by side on one display screen is increased, the information amount of the ECG waveforms is increased. Therefore, the operator such as a medical person can perform adequate diagnosis based on the plurality of displayed ECG waveforms. On the other hand, as the number of ECG waveforms that are displayed side by side on one display screen is increased, the display intervals between adjacent ECG waveforms are narrowed, and therefore parts (particularly, steep portions such as QRS waveforms) of adjacent ECG waveforms overlap each other. As a result, the visibilities of the ECG waveforms are lowered. Also in the case where the baseline fluctuates, such as in the case of drift, furthermore, a same or similar phenomenon occurs.

JP-A-4-150832 discloses a technique in which, in order to prevent adjacent ECG waveforms from partly overlapping each other, a plurality of ECG waveforms are displayed side by side on one display screen in a form where each of the waveforms is compressed. In the technique disclosed in JP-A-4-150832, since each of the ECG waveforms is compressed, the visibility of each ECG waveform is lowered, and therefore an enlarged display window for enlarging and displaying each ECG waveform is disposed.

### SUMMARY

In the technique disclosed in JP-A-4-150832, however, ECG waveforms are compressed, and therefore it is difficult to adequately analyze the ECG waveform which is a selection object, while viewing the whole ECG waveforms that are displayed side by side on one display screen. To comply with this, a part of the ECG waveform which is a selection object must be enlarged and displayed using the enlarged display window. When the enlarged display window is displayed on the display screen, however, parts of vital sign waveforms displayed on the display screen are hidden by the enlarged display window, and cannot be viewed by the operator.

An aspect of the present disclosure provides a vital sign waveform display apparatus and vital sign waveform display method in which the visibility of a vital sign waveform selected by the operator can be improved without reducing the number of vital sign waveforms that are displayed side by side on a display screen.

Another aspect of the present disclosure provides a program for realizing the vital sign waveform display method, and a computer readable storage medium storing the program.

According to a first aspect of the present disclosure, a vital sign waveform display apparatus comprises:

an acquiring section configured to acquire vital sign waveform data representing a plurality of vital sign waveforms;

a generating section configured to generate the plurality of vital sign waveforms, based on the acquired vital sign waveform data; and

a display controller configured to display the generated vital sign waveforms side by side on a display screen, wherein

the display controller highlights a first vital sign waveform when the first vital sign waveform is selected from the vital sign waveforms displayed on the display screen, and then

the display controller highlights the first vital sign waveform and a second vital sign waveform when the second vital sign waveform is selected from the vital sign waveforms displayed on the display screen.

According to a second aspect of the present disclosure, a vital sign waveform display method comprises:

(a) acquiring vital sign waveform data representing a plurality of vital sign waveforms;

(b) generating the plurality of vital sign waveforms, based on the acquired vital sign waveform data;

(c) displaying the generated vital sign waveforms side by side on a display screen;

(d) highlighting a first vital sign waveform when the first vital sign waveform is selected from the vital sign waveforms displayed on the display screen; and

(e) highlighting the first vital sign waveform and a second vital sign waveform when the second vital sign waveform is selected from the vital sign waveforms displayed on the display screen, after the step (d).

According to a third aspect of the present disclosure, there is provided a program causing a computer to perform operations comprising:

(a) acquiring vital sign waveform data representing a plurality of vital sign waveforms;

(b) generating the plurality of vital sign waveforms, based on the acquired vital sign waveform data;

(c) displaying the generated vital sign waveforms side by side on a display screen;

(d) highlighting a first vital sign waveform when the first vital sign waveform is selected from the vital sign waveforms displayed on the display screen; and

(e) highlighting the first vital sign waveform and a second vital sign waveform when the second vital sign waveform is selected from the vital sign waveforms displayed on the display screen, after the operation (d).

According to the present disclosure, it is possible to provide a vital sign waveform display apparatus in which the visibility of a vital sign waveform selected by the operator can be improved without reducing the number of vital sign waveforms that are displayed side by side on the display screen.

### BRIEF DESCRIPTION OF DRAWINGS

FIG. 1 is a hardware configuration diagram illustrating an electrocardiogram waveform display apparatus of an embodiment of the invention.

FIG. 2 is a functional block diagram of a controller.

FIG. 3 is a flowchart illustrating an electrocardiogram waveform display method of the embodiment of the invention.

FIG. 4 illustrates a display screen on which a plurality of electrocardiogram waveforms are displayed side by side.

FIG. 5 illustrates a display screen on which an electrocardiogram waveform V2 is highlighted.

FIG. 6 illustrates a display screen on which the electrocardiogram waveform V2 and an electrocardiogram waveform aVR are highlighted.

### DESCRIPTION OF EMBODIMENTS

An embodiment of the present disclosure will be described with reference to the drawings. In the description of the embodiment, description of components which are denoted by the same reference numerals as those designating components that have been already described will be omitted for the sake of convenience of description.

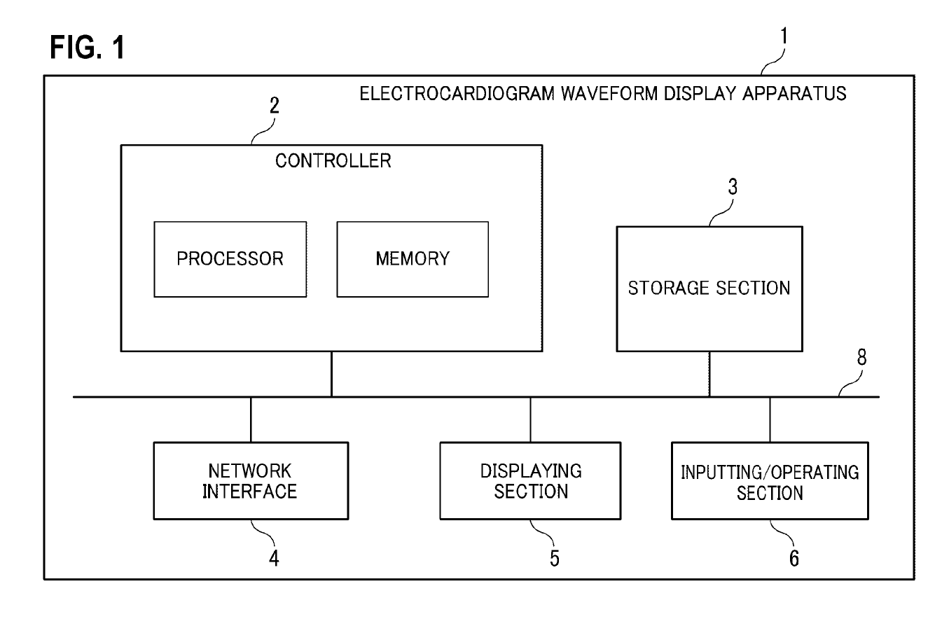

FIG. 1 is a hardware configuration diagram of an electrocardiogram waveform display apparatus 1 (an example of the vital sign waveform display apparatus) of the embodiment of the invention (hereinafter, referred to merely as “the embodiment”). As illustrated in FIG. 1, the electrocardiogram waveform display apparatus 1 (hereinafter, referred to merely as “the display apparatus 1”) may include a controller 2, a storage section 3, a network interface 4, a displaying section 5, and an inputting/operating section 6. These are communicably connected to one another through a bus 8.

The display apparatus 1 may be a dedicated apparatus for analyzing an electrocardiogram waveform (an example of the vital sign waveform), a personal computer, a wearable device such as a smart phone, a tablet, or an Apple Watch, or a patient monitor.

The controller 2 may include a memory and a processor. For example, the memory is configured by a ROM (Read Only Memory) in which various programs and the like are stored, a RAM (Random Access Memory) having a plurality of work areas in which various programs that are to be executed by the processor, and the like are to be stored, and the like. For example, the processor is a CPU (Central Processing Unit), and configured to develop designated programs in the various programs incorporated in the ROM, in the RAM, and execute various processes in cooperation with the RAM.

Particularly, the processor may develop an ECG waveform display program which will be described later, in the RAM, and cooperate with the RAM to execute the program, thereby enabling the controller 2 to control various operations of the display apparatus 1. The controller 2 and the ECG waveform display program will be described in detail later.

The storage section 3 is a storage device such as an HDD (Hard Disk Drive), an SSD (Solid State Drive), or a flash memory, and configured to store programs and various data. The ECG waveform display program (an example of the vital sign waveform display program) may be incorporated in the storage section 3. Moreover, ECG waveform data acquired by an ECG sensor which is not illustrated may be stored in the storage section 3. The ECG sensor is configured to measure weak electrical signals generated by the heart of the patient, thereby acquiring ECG waveform data. Here, the ECG waveform data mean a plurality of ECG waveforms (12-lead ECG waveforms or the like) having heartbeat waveforms (such as QRS waveforms) which are continuously generated on the time axis.

The network interface 4 is configured to connect the display apparatus 1 to a communication network which is not illustrated. The communication network is a LAN (Local Area Network), a WAN (Wide Area Network), the Internet, or the like. For example, the ECG waveform display program and the ECG waveform data may be acquired from a computer placed on the communication network through the network interface 4.

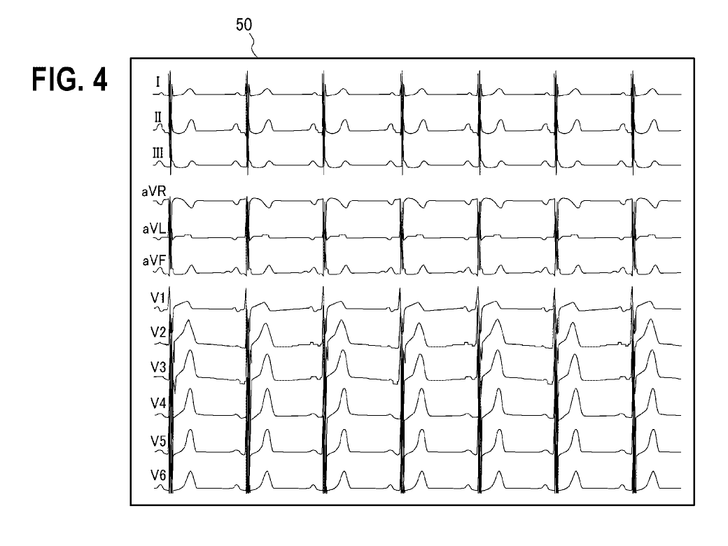

The displaying section 5 is a display device such as a liquid crystal display or an organic EL display. The displaying section 5 has a display screen 50 which will be described later. A plurality of ECG waveforms which are generated by the controller 2 are displayed side by side on the display screen 50 of the displaying section 5 (see FIG. 4 and the like).

The inputting/operating section 6 receives an input operation performed by the operator who operates the display apparatus 1, and is configured to output an instruction signal in accordance with the input operation. For example, the inputting/operating section 6 is a touch panel which is overlaid on the displaying section 5, operation buttons which are attached to a housing, a mouse, a keyboard, or the like.

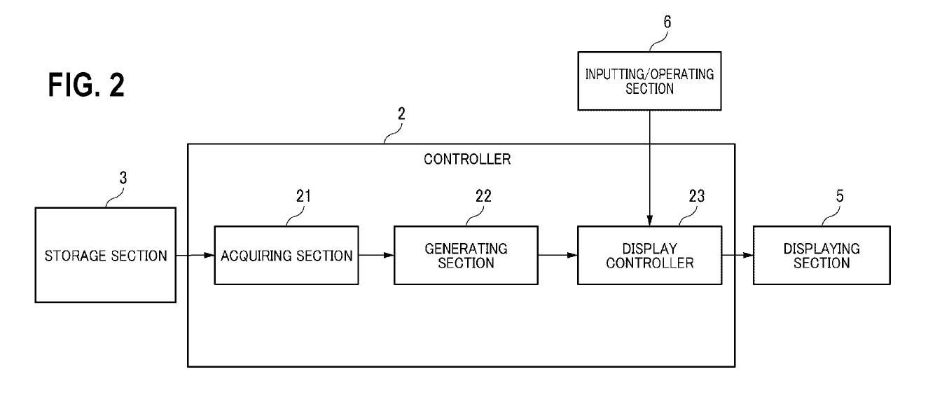

FIG. 2 is a functional block diagram of the controller 2 of the display apparatus 1. As illustrated in FIG. 2, the controller 2 may include an acquiring section 21, a generating section 22, and a display controller 23. The acquiring section 21 is configured to acquire ECG waveform data (an example of the vital sign waveform data) representing a plurality of ECG waveforms (an example of the vital sign waveform), from the storage section 3. The generating section 22 is configured to generate a plurality of ECG waveforms and a plurality of waveform display labels based on the ECG waveform data acquired by the acquiring section 21. The plurality of waveform display labels correspond to one of the ECG waveforms. The display controller 23 is configured to display the plurality of ECG waveforms generated by the generating section 22 side by side on the display screen 50 of the displaying section 5 (see FIG. 4). Moreover, the display controller 23 is configured to display the plurality of waveform display labels (see the left side of FIG. 4) on the display screen 50. In accordance with an input operation of the operator such as a medical person, furthermore, the display controller 23 is configured to highlight an ECG waveform(s) displayed on the display screen 50. Particularly, when the operator selects a predetermined waveform display label, the display controller 23 is configured to highlight the ECG waveform corresponding to the selected waveform display label.

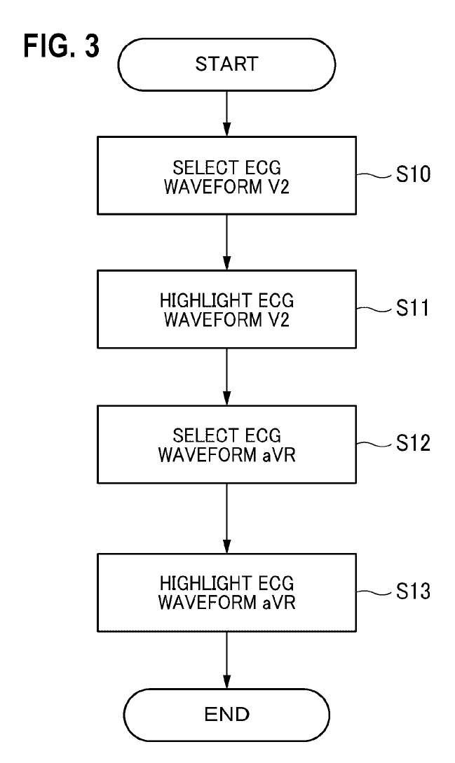

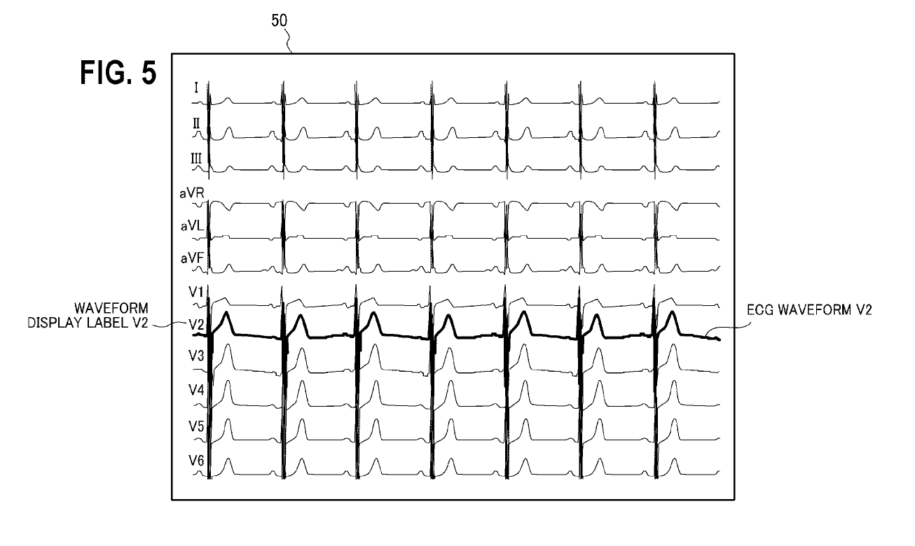

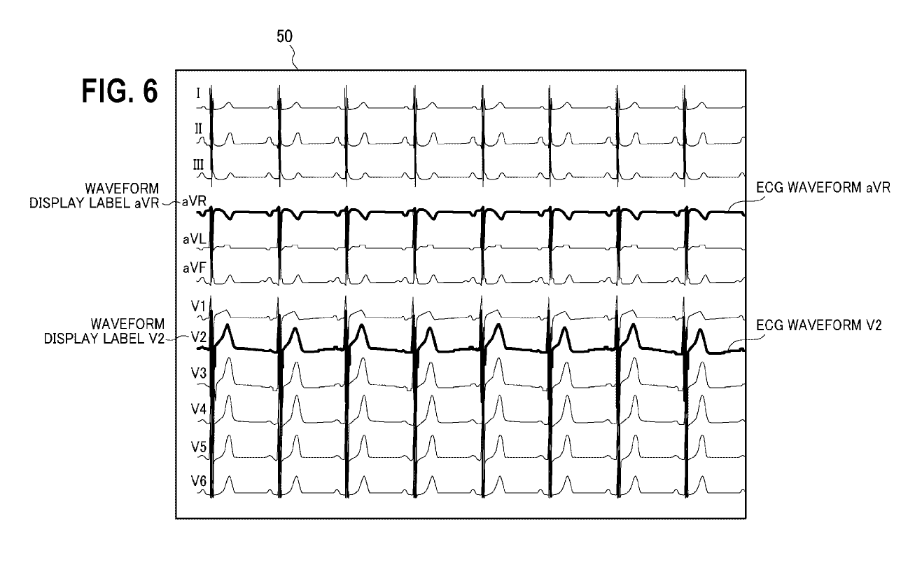

Next, the ECG waveform display method (an example of the vital sign waveform display method) of the embodiment will be described with reference to FIGS. 3 to 6. FIG. 3 is a flowchart illustrating the ECG waveform display method. FIG. 4 illustrates the display screen 50 on which a plurality of ECG waveforms are displayed side by side in the longitudinal direction. FIG. 5 illustrates the display screen 50 on which an ECG waveform V2 is highlighted. FIG. 6 illustrates the display screen 50 on which the ECG waveform V2 and an ECG waveform aVR are highlighted.

On the display screen 50, as illustrated in FIG. 4, 12 ECG waveforms (i.e., ECG waveform I corresponding to lead I, ECG waveform II corresponding to lead II, ECG waveform III corresponding to lead III ECG waveform aVR corresponding to lead aVR, ECG waveform aVL corresponding to lead aVL, ECG waveform aVF corresponding to lead aVF, and ECG waveforms V1 to V6 corresponding to lead V1 to V6, respectively) are displayed. Moreover, steep portions of QRS waveforms of ECG waveforms which are adjacent to each other in the longitudinal direction overlap each other, and therefore the visibilities of the ECG waveforms are lowered. Consequently, the operator hardly analyzes the ECG waveforms illustrated in FIG. 4 with a sufficiently high accuracy.

In step S10, as illustrated in FIG. 3, the operator selects the ECG waveform V2 (first vital sign waveform) which is the analysis object, through the inputting/operating section 6. When the waveform display label V2 (see FIG. 5) corresponding to the ECG waveform V2 is designated by an operation performed on the inputting/operating section 6 (for example, the waveform display label V2 is clicked), specifically, the inputting/operating section 6 generates an instruction signal indicating the designation of the waveform display label V2, and transmits the instruction signal to the display controller 23. In accordance with the received instruction signal, thereafter, the display controller 23 determines that the ECG waveform V2 is selected by the operator, and highlights the ECG waveform V2 displayed on the display screen 50 (step S11). In this point, the display controller 23 highlights the ECG waveform V2 displayed on the display screen 50, without displaying the ECG waveform V2 in an enlarged display window which is different from than the display screen 50. As illustrated in FIG. 5, specifically, the display controller 23 highlights the electrocardiogram waveform V2, by changing the color of the electrocardiogram waveforms other than the ECG waveform V2 to a color (pale color) which is higher in brightness than the color of the ECG waveform V2. The highlighted display of the selected ECG waveform means that the selected ECG waveform is highlighted with respect to the other ECG waveforms. The selected ECG waveform is highlighted not only by changing the display mode (the display color or the like) of the selected ECG waveform, but also by, as in the embodiment, changing the display modes (the display colors or the like) of the ECG waveforms other than the selected ECG waveform.

In order to compare and analyze the ECG waveform V2 and the ECG waveform aVR, next, the operator selects the ECG waveform aVR (second vital sign waveform) through the inputting/operating section 6 (step S12). When the waveform display label aVR (see FIG. 6) corresponding to the ECG waveform aVR is designated by an operation performed on the inputting/operating section 6 (for example, a cursor is moved to the position of the waveform display label aVR), specifically, the inputting/operating section 6 generates an instruction signal indicating the designation of the waveform display label aVR, and transmits the instruction signal to the display controller 23. In accordance with the received instruction signal, thereafter, the display controller 23 determines that the ECG waveform aVR is selected by the operator, and highlights the electrocardiogram waveform aVR displayed on the display screen 50 (step S13). When the ECG waveform aVR is selected by the operator after step S11, as described above, the display controller 23 highlights the ECG waveform V2 and the ECG waveform aVR. In this regard, the display controller 23 highlights the ECG waveform aVR displayed on the display screen 50, without displaying the ECG waveform aVR in an enlarged display window which is different from the display screen 50. As illustrated in FIG. 6, specifically, the display controller 23 highlights the ECG waveform aVR, by restoring the color of the ECG waveform aVR from the color (the color after the change which is illustrated in FIG. 5) having a high brightness to the color (the color before the change which is illustrated in FIG. 4) having a low brightness. It should be noted that, in the figures (FIGS. 5 and 6), the ECG waveforms each having a low brightness are indicated by thin lines while the ECG waveform having a high brightness is indicated by a thick line.

When the operator moves the cursor from the position of the vital sign display label aVR to that of a vital sign display label III by using the inputting/operating section 6, moreover, the inputting/operating section 6 generates an instruction signal indicating the designation of the vital sign display label III, and transmits the instruction signal to the display controller 23. In accordance with the received instruction signal, thereafter, the display controller 23 determines that the ECG waveform III is selected by the operator, and highlights the ECG waveform III, by restoring the color of the ECG waveform aVR to the color (the color after the change which is illustrated in FIG. 5) having a high brightness, and restoring the color of the ECG waveform III to the color (the color before the change which is illustrated in FIG. 4) having a low brightness. In this way, the display controller 23 highlights the ECG waveform V2 and the ECG waveform III. As a result, the operator can compare and analyze the ECG waveform V2 and the ECG waveform III.

According to the embodiment, the ECG waveform V2 which is selected by the operator is highlighted, and the ECG waveform aVR which is selected by the operator is highlighted. Therefore, the display apparatus 1 can be provided in which the visibilities of the ECG waveforms V2, aVR which are selected by the operator can be improved without reducing the number of plural ECG waveforms that are displayed side by side on the display screen 50 (in other words, in a state where all the ECG waveforms that are displayed on the display screen 50 are visible). Moreover, the operator such as a medical person can compare and analyze the ECG waveform V2 and the electrocardiogram waveform aVR while referring to other ECG waveforms as required, and therefore rapidly perform adequate diagnosis.

According to the embodiment, since a plurality of ECG waveforms are displayed side by side on the display screen 50, moreover, parts (steep portions such as QRS waveforms) of adjacent ECG waveforms overlap each other, and therefore the visibilities of the ECG waveforms are easily lowered. Therefore, the embodiment is particularly effective in the case where a plurality of ECG waveforms are displayed side by side on the display screen 50.

According to the embodiment, furthermore, the colors of the electrocardiogram waveforms other than the ECG waveform V2 are changed to a color which is higher in brightness than the color of the ECG waveform V2, and the color of the ECG waveform aVR is restored to that before change (the color shown in FIG. 4). Therefore, the visibilities of the ECG waveforms V2, aVR which are selected by the operator can be improved.

According to the embodiment, furthermore, an ECG waveform can be selected by designating the corresponding waveform display label without directly designating the ECG waveform. As compared with the case where an ECG waveform is directly designated, therefore, a desired ECG waveform can be easily selected, and hence the operability of the display apparatus 1 can be improved.

In the embodiment, in order to highlight the ECG waveform V2, the colors of the ECG waveforms other than the ECG waveform V2 are changed to a color which is higher in brightness than the color of the ECG waveform V2. Alternatively, the colors of the ECG waveforms other than the ECG waveform V2 may be changed to a color which is different from the color of the ECG waveform V2. Particularly, the hues of the ECG waveforms other than the ECG waveform V2 may be changed to a hue which is different from the hue of the ECG waveform V2. Moreover, the saturations of the ECG waveforms other than the ECG waveform V2 may be changed to a saturation which is different from the saturation of the ECG waveform V2. Also in this case, similarly, the ECG waveform V2 can be highlighted. As described above, the colors (the hues, the brightnesses, or the saturations) of the ECG waveforms other than the ECG waveform V2 are changed to a color which is different from the color (the hue, the brightness, or the saturation) of the ECG waveform V2, and the color of the ECG waveform aVR is returned to that before change, whereby the visibilities of the ECG waveforms V2, aVR which are selected by the operator can be improved.

Alternatively, the display controller 23 may highlight the ECG waveform V2 by reducing the line widths of the ECG waveforms other than the ECG waveform V2 as compared with the line width of the ECG waveform V2. Furthermore, the display controller 23 may display the ECG waveforms other than the ECG waveform V2 by dotted lines or broken lines, thereby highlighting the ECG waveform V2.

The display controller 23 may change the color (the hue, the brightness, or the saturation) or line width of the ECG waveform V2, thereby highlighting the ECG waveform V2, and the color (the hue, the brightness, or the saturation) or line width of the ECG waveform aVR, thereby highlighting the ECG waveform aVR. Also in this case, similarly, the visibilities of the ECG waveforms V2, aVR which are selected by the operator can be improved in the state where all the ECG waveforms that are displayed on the display screen 50 are visible.

In order to realize the display apparatus I of the embodiment in software, the ECG waveform display program may be installed in the storage section 3 or the ROM in advance. Alternatively, the ECG waveform display program may be stored on a computer readable storage medium such as a magnetic disk (an HDD or a floppy disk), an optical disk (a CD-ROM, a DVD-ROM, a Blu-ray disk, or the like), a magneto-optical disk (an MD or the like), or a flash memory (an SD card, a USB memory, an SSD, or the like). In the alternative, when the storage medium is connected to the display apparatus 1, the ECG waveform display program which is stored on the storage medium is incorporated into the storage section 3. Then, the program incorporated in the storage section 3 is loaded into the RAM, the processor executes the loaded program, and as a result the controller 2 executes the various processes illustrated in FIG. 2. In other words, when the program is executed by the processor, the controller 2 realizes the functions of: acquiring electrocardiogram waveform data indicating a plurality of electrocardiogram waveforms; generating a plurality of ECG waveforms based on the acquired ECG waveform data; displaying the plurality of generated ECG waveforms side by side on the display screen 50; when the ECG waveform V2 in the plurality of ECG waveforms displayed on the display screen 50 is selected by the operator, highlighting the ECG waveform V2; and, when the ECG waveform aVR in the plurality of ECG waveforms displayed on the display screen 50 is selected by the operator, highlighting the electrocardiogram waveform V2 and the ECG waveform aVR.

Alternatively, the ECG waveform display program may be downloaded from a computer on a communication network, through the network interface 4. Also in the alternative, similarly, the downloaded program is incorporated into the storage section 3.

Although the embodiment of the present disclosure has been described above, the technical scope of the invention should not be limitedly interpreted based on the description of the embodiment. The embodiment is merely an example. It should b e understood by those skilled in the art that change can be made on the embodiment variously within the scope of the claimed invention. The technical scope of the invention should be defined based on the scope of the claimed invention and the scope of any equivalent thereto.

Although the embodiment in which the 12 ECG waveforms (12-lead ECG) are displayed side by side on the display screen 50 has been described, the number of ECG waveforms is not limited to 12, and various numbers of ECG waveforms may be displayed side by side on the display screen 50. Moreover, although the embodiment in which two selected ECG waveforms are highlighted has been described, the display controller 23 may highlight only one ECG waveform, or three or more ECG waveforms. When three ECG waveforms 1, aVF, V5 are selected by an operation through the inputting/operating section 6, for example, the display controller 23 is configured to highlight the three ECG waveforms I, aVF, V5.

Although the embodiment in which the display apparatus 1 for displaying ECG waveforms is an example of the vital sign waveform display apparatus of the present invention has been described, the vital sign waveform display apparatus of the invention is not limited to the ECG waveform display apparatus 1. For example, the vital sign waveform display apparatus of the present invention may be a patient monitor for displaying vital sign waveforms such as the brain wave, the pulse wave, the ECG, the electromyogram, and the expiration. Also in this case, when a selected vital sign waveform is highlighted, similarly, the visibility of the selected vital sign waveform can be improved without reducing the number of vital sign waveforms that are displayed side by side on the display screen.

What is claimed is:

1. A vital sign waveform display apparatus comprising:
an acquiring section configured to acquire vital sign waveform data representing a plurality of vital sign waveforms;
a generating section configured to generate the plurality of vital sign waveforms, based on the acquired vital sign waveform data; and
a display controller configured to display the generated vital sign waveforms side by side on a display screen, wherein
the display controller highlights a first vital sign waveform when the first vital sign waveform is selected from the vital sign waveforms displayed on the display screen, and then
the display controller highlights the first vital sign waveform and a second vital sign waveform when the second vital sign waveform is selected from the vital sign waveforms displayed on the display screen.

2. The vital sign waveform display apparatus of claim 1, wherein the vital sign waveforms are electrocardiogram waveforms.

3. The vital sign waveform display apparatus of claim 1, wherein
the display controller is configured to:
change colors of the vital sign waveforms other than the first vital sign waveform to a color which is different from a color of the first vital sign waveform so as to highlight the first vital sign waveform; and
restore a color of the second vital sign waveform to a color before the change so as to highlight the second vital sign waveform.

4. The vital sign waveform display apparatus of claim 3, wherein
the display controller is configured to change the colors of the vital sign waveforms other than the first vital sign waveform to a color which is higher in brightness than the color of the first vital sign waveform so as to highlight the first vital sign waveform.

5. The vital sign waveform display apparatus of claim 1, wherein the display controller is configured to:
change a color or line width of the first vital sign waveform so as to highlight the first vital sign waveform; and
change a color or line width of the second vital sign waveform so as to highlight the second vital sign waveform.

6. The vital sign waveform display apparatus of claim 1, wherein
the display controller is configured to display a plurality of waveform display labels on the display screen, wherein each of the waveform display labels corresponds to one of the vital sign waveforms,
a first vital sign waveform corresponding to the first vital sign waveform is selected when the first waveform display label is designated by an operator, and
a second waveform display label corresponding to the second vital sign waveform is selected when the second waveform display label is designated by an operator.

7. A vital sign waveform display method comprising:
(a) acquiring vital sign waveform data representing a plurality of vital sign waveforms;
(b) generating the plurality of vital sign waveforms, based on the acquired vital sign waveform data;
(c) displaying the generated vital sign waveforms side by side on a display screen;
(d) highlighting a first vital sign waveform when the first vital sign waveform is selected from the vital sign waveforms displayed on the display screen; and
(e) highlighting the first vital sign waveform and a second vital sign waveform when the second vital sign waveform is selected from the vital sign waveforms displayed on the display screen, after the step (d).

8. The method of claim 7, wherein the vital sign waveforms are electrocardiogram waveforms.

9. The method of claim 7, wherein
the step (d) comprises changing colors of the vital sign waveforms other than the first vital sign waveform to a color which is different from a color of the first vital sign waveform so as to highlight the first vital sign waveform, and
the step (e) comprises restoring a color of the second vital sign waveform to a color before the change so as to highlight the second vital sign waveform.

10. The method of claim 9, wherein
the step (d) comprises changing the colors of the vital sign waveforms other than the first vital sign waveform to a color which is higher in brightness than the color of the first vital sign waveform so as to highlight the first vital sign waveform.

11. The method of claim 7, wherein
the step (d) comprises changing a color or line width of the first vital sign waveform so as to highlight the first vital sign waveform, and
the step (e) comprises changing a color or line width of the second vital sign waveform so as to highlight the second vital sign waveform.

12. The method of claim 7, wherein
the step (c) comprises displaying a plurality of waveform display labels on the display screen, wherein each of the waveform display labels corresponds to one of the vital sign waveforms,
the step (d) comprises selecting a first vital sign waveform corresponding to the first vital sign waveform when the first waveform display label is designated by an operator, and
the step (e) comprises selecting a second waveform display label corresponding to the second vital sign waveform when the second waveform display label is designated by an operator.

A non-transitory computer-readable medium storing a program causing a computer to perform operations comprising:
(a) acquiring vital sign waveform data representing a plurality of vital sign waveforms;
(b) generating the plurality of vital sign waveforms, based on the acquired vital sign waveform data;
(c) displaying the generated vital sign waveforms side by side on a display screen;
(d) highlighting a first vital sign waveform when the first vital sign waveform is selected from the vital sign waveforms displayed on the display screen; and
(e) highlighting the first vital sign waveform and a second vital sign waveform when the second vital sign waveform is selected from the vital sign waveforms displayed on the display screen, after the operation (d).

14. (canceled)
